(19) United States
(12) Patent Application Publication
(10) Pub. No.: US 2017/0071668 A1
(43) Pub. Date: Mar. 16, 2017

(54) RADIOFREQUENCY ABLATION CATHETER AND THERAPY DEVICE INCLUDING THE RADIOFREQUENCY ABLATION CATHETER

(71) Applicant: ANGEL MEDICAL TECHNOLOGY (NANJING) CO., LTD., Nanjing (CN)

(72) Inventors: Chi Ming CHAN, Guangzhou (CN); Linhua LI, Guangzhou (CN)

(73) Assignee: ANGEL MEDICAL TECHNOLOGY (NANJING) CO., LTD., Nanjing (CN)

(21) Appl. No.: 15/361,952

(22) Filed: Nov. 28, 2016

Related U.S. Application Data
(63) Continuation of application No. PCT/CN2015/086839, filed on Aug. 13, 2015.

(30) Foreign Application Priority Data
Jul. 2, 2015 (CN) 201510383506.X

(51) Int. Cl. A61B 18/14

(52) U.S. Cl. CPC A61B 18/1492; A61B 2018/00577

(57) ABSTRACT

A radiofrequency ablation catheter includes an electrode ablation head (10), a Positive Temperature Coefficient (PTC) thermistor (20), and an inner conductor (30), where the electrode ablation head (10) is disposed at an end portion of the inner conductor (30), the PTC thermistor is disposed between the electrode ablation head (10) and the inner conductor (30), and the inner conductor (30) is connected to a cable plug component (40). The radiofrequency ablation catheter and a therapy device including the radiofrequency ablation catheter can precisely control the ablation temperature by means of PTC, such that upon coagulation of tissues, the ablation catheter is not adhered to the tissues, thus avoiding the risk of complications such as rebleeding.

### CROSS REFERENCE TO RELATED APPLICATIONS

This application is a continuation of PCT/CN2015/086839 filed Aug. 13, 2015, which claims priority to CN 201510383506.X filed Jul. 2, 2015, both of which are incorporated herein by reference.

### TECHNICAL FIELD

The present invention relates to the technical field of medical radiofrequency ablation, and in particular, to a radiofrequency ablation catheter and a therapy device including the radiofrequency ablation catheter.

### RELATED ART

Minimally invasive ablation is a development trend of the medical industry, and thermal ablation has become a mainstream ablation treatment means because it has definite effects. The thermal ablation mainly includes three types: microwave, laser, and radiofrequency, and each type of thermal ablation means has its own advantages and disadvantages. All the three types of minimally invasive thermal ablation treatment means are implemented via various ablation electrodes. In terms of structures, the ablation electrodes generally can be classified into hard electrodes and flexible electrodes, and the vascular intervention treatment, for example, treatment for diseases such as varicosity, arrhythmia, and angiemphraxis, mainly relies on flexible electrodes (hard electrodes may be used in superficial vascular intervention), while non-vascular intervention treatment mainly relies on direct or indirect puncture of hard electrodes, for example, percutaneous puncture ablation is used to treat liver cancer, uterine fibroid, lung cancer, thyroid nodule, and the like (where laser ablation, whether it is vascular intervention or non-vascular intervention, needs guidance of a catheter due to the particularity of a laser fiber structure). In the field of minimally invasive therapy, catheter-type ablation electrodes have broad space for development, where radio frequency catheter electrodes have incomparable advantages.

Leg varicosity is not only painful but also affects the appearance. Varicosity is the most common disease in the venous system. Varicosity occurs mainly because people hold a same position with barely any change in a long period of time, causing blood to accumulate at legs. Year after year, the venous valve is damaged and an excessively high venous pressure is generated, resulting in varicosity.

Varicosity mostly appears at legs. Red or blue tor tuousvessels like a spider web or an earthworm or burl-like lump nodules appear on the skin. The vein expands and swells abnormally, and the varix occurs. As human body does not have a self-healing mechanism for valves, the varicosity is an irreversible phenomenon.

Traditional high ligation and extraction as well as varicose vein stripping for great saphenous veins are classic surgeries for treating lower extremity varicosity. However, these surgeries have disadvantages such as a long hospital stay, a large number of incisions, and obvious postoperative scars that affect the appearance and especially, a lot of female patients who want their legs to be beautiful are afraid of such surgeries, and even delay the treatment. With the continuous improvement of living standards, some patients, especially young female patients, have higher requirements on therapeutic levels. They not only require treatment of the disease and prevention of complications, but also require the number of incisions to be reduced, the incisions to be small or even invisible, and the appearance is not affected after the surgery.

The traditional vein stripping or surgical therapy requires patients to rest for 3 weeks after the surgery, and leaves large scars; another non-surgical vascular sclerosis therapy is to inject radioactive materials to lock the varicose veins, which is harmful to a normal body in some degree, and the varicosity may recur.

Radiofrequency ablation vein closure is a new method for treating great saphenous varicosity. It is carried out in recent years in Europe, America, and other countries, and has achieved satisfactory results. Currently, a number of multi-center prospective randomized controlled clinical studies have demonstrated that endovenous radiofrequency closure treatment of great saphenous veins is safe and effective.

The existing radiofrequency ablation technique usually adopts an impedance control manner and a temperature control manner to control the ablation degree.

The impedance control adopts impedance of 400-500 ohms, but it is difficult to flexibly adjust the specific impedance. If the impedance is too small, the ablation will be ended earlier than expected, and the ablation is incomplete, while if the impedance is too large, it easily leads to tissue adhesion.

The Positive Temperature Coefficient (PTC) thermistor is a typical semiconductor resistor with temperature sensitivity, and when the temperature exceeds a certain value, its electrical resistance increases step-wise as the temperature increases. The term “PTC” is an abbreviation of Positive Temperature Coefficient, and generally refers to semiconductor materials or devices with a very large positive temperature coefficient. The commonly mentioned PTC refers to a positive temperature coefficient thermistor, which is referred to as PTC thermistor for short. The electrical resistance of the positive temperature coefficient thermistor increases step-wise as the temperature of the PTC thermistor increases. The higher the temperature is, the higher the electrical resistance is.

The present invention makes use of characteristics of a PTC thermistor to design an intelligent-controlled novel ablation catheter.

### SUMMARY

An objective of the present invention is to provide a radiofrequency ablation catheter and a therapy device including the radiofrequency ablation catheter, to overcome disadvantages of the impedance control and temperature control by using characteristics of a PTC thermistor.

Technical solutions of the present invention are described as follows:

A radiofrequency ablation catheter is provided, including an electrode ablation head 10, a PTC thermistor 20, and an inner conductor 30, where the electrode ablation head 10 is disposed at an end portion of the inner conductor 30, the PTC thermistor is disposed between the electrode ablation head 10 and the inner conductor 30, and the inner conductor 30 is connected to a cable plug component 40.

The electrode ablation head 10 is a tubular body, and a tail end thereof is smooth and arc-shaped.

A material of the electrode ablation head 10 is 304 medical stainless steel.

A critical temperature of the PTC thermistor 20 is 80° C. to 120° C.

Another radiofrequency ablation catheter according to the present invention includes an outer connector 1, a PTC thermistor 2, an inner conductor 3, an insulated conduit 4, where the insulated conduit 4 is rod-like, and a tail end thereof is arc-shaped; an outer surface of the insulated conduit 4 is provided with the cylindrical outer connector 1; an inner wall of the outer connector 1 is provided with the PTC thermistor 2; an inner cable 7 is disposed inside the insulated conduit 4; the inner cable 7 and the outer connector 1 are connected inside the insulated conduit 4 through the PTC thermistor 2; the radiofrequency ablation catheter further includes: a cable plug component 6 which is connected to the inner cable 7, where the inner cable 7 is connected to the cable plug component 6 via the interior of a Y-shaped three-way insulation tube 5.

A material of the first outer connector 1 is 304 medical stainless steel.

A material of the insulated conduit 4 is polytetrafluoroethylene (PTFE) or Teflon.

A third radiofrequency ablation catheter according to the present invention includes a first outer connector 1, a PTC thermistor 2, a second outer connector 3, an insulated conduit 4, a first inner cable 5, and a second inner cable 6, where the insulated conduit 4 is rod-like, and a tail end thereof is arc-shaped; an outer surface of the insulated conduit 4 is provided with the cylindrical first outer connector 1 and the cylindrical second outer connector 3 respectively; an inner wall of the first outer connector 1 is provided with the PTC thermistor 2; the first inner cable 5 and the second inner cable 6 are disposed inside the insulated conduit 4; the second outer connector 3 and the first inner cable 5 are electrically connected inside the insulated conduit 4; and the second inner cable 6 and the first outer connector 1 are connected inside the insulated conduit 4 via the PTC thermistor 2.

A fourth therapy device including a radiofrequency ablation catheter according to the present invention includes a cable plug component 8 which is connected to a first inner cable 5 and a second inner cable 6, where the first inner cable 5 and the second inner cable 6 are connected to the cable plug component 8 via the interior of a Y-shaped three-way insulation tube 7.

The present invention achieves the following beneficial effects:

A radiofrequency ablation catheter and a therapy device including the radiofrequency ablation catheter according to the present invention are convenient to use, eliminate the varicosity instantly, barely cause allergies, are efficient, and do not cause necrosis; the pigmentation does not last long, and the treatment is fast (the course of treatment lasts 10-15 minutes at most); for 300-400 pulses, 80-100 cm of varicose veins can be eliminated in a single course of treatment.

The radiofrequency ablation catheter and the therapy device including the radiofrequency ablation catheter according to the present invention can be used for various types of varicosities (small varicosity), cover all positions and micro-sclerose unreachable small areas (<0.3 mm), and are applicable to all types of skins.

The new ablation device according to the present invention makes use of the characteristics of PTC thermistor. In combination with a radio frequency generator, the PTC thermistor is designed for heat treatment of target biological tissues. Such heat treatment may be carried out within the temperature range of 80° C. to 120° C., and preferably 100° C., such that upon coagulation of the tissues, the ablation catheter is not adhered to the tissues, thus avoiding the risk of complications such as rebleeding.

The new ablation catheter of the present invention makes use of such characteristic of the PTC thermistor: the electrical resistance of the PTC thermistor increases as its temperature rises; besides, the PTC thermistor in the present invention is distributed in the entire ablation region. Therefore, in contrast to the existing total on-off, the present invention can achieve partial on-off, till all the tissues in the ablation region reach the predetermined ablation temperature, thus realizing precise control over the ablation, so that all target positions can be completely ablated. In addition, once the tissues in the ablation region reach the predetermined temperature, the PTC thermistors are turned off, thus avoiding excessive damage to the tissues.

The radiofrequency ablation catheter according to the present invention can be used for clearing and dilating blood vessels. The radiofrequency ablation catheter according to the present invention may be catheters of three specifications: 3F, 5F, and 8F. Radio frequency energy is transferred to the electrode at the tip of the catheter, and therefore the heat is transferred into the blood vessel.

The radiofrequency ablation catheter according to the present invention can be further used for tissue coagulation in a gastrointestinal tract, digestive tract (including the bile duct) endoscopic surgery or a percutaneous surgery (the radio frequency energy is transferred to the electrode at the tip of the catheter to heat tissues surrounding the catheter).

### BRIEF DESCRIPTION OF THE DRAWINGS

FIG. 1 is a schematic structural diagram of Embodiment 1 of the present invention;

FIG. 2 is a schematic structural diagram of Embodiment 2 of the present invention;

FIG. 3 is a schematic partial enlarged diagram of FIG. 2;

FIG. 4 is a schematic structural diagram of Embodiment 3 of the present invention; and

FIG. 5 is a schematic structural diagram of Embodiment 4 of the present invention.

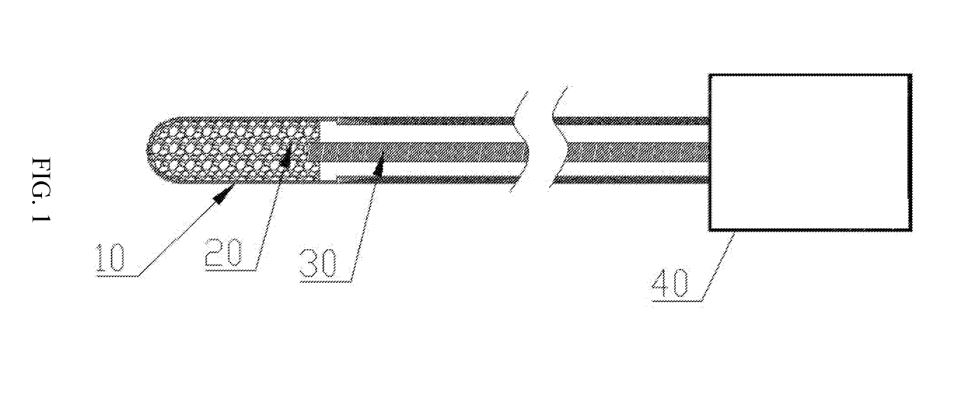

In FIG. 1: 10 represents an electrode ablation head, 20 represents a PTC thermistor, 30 represents an inner conductor, and 40 represents a cable plug component.

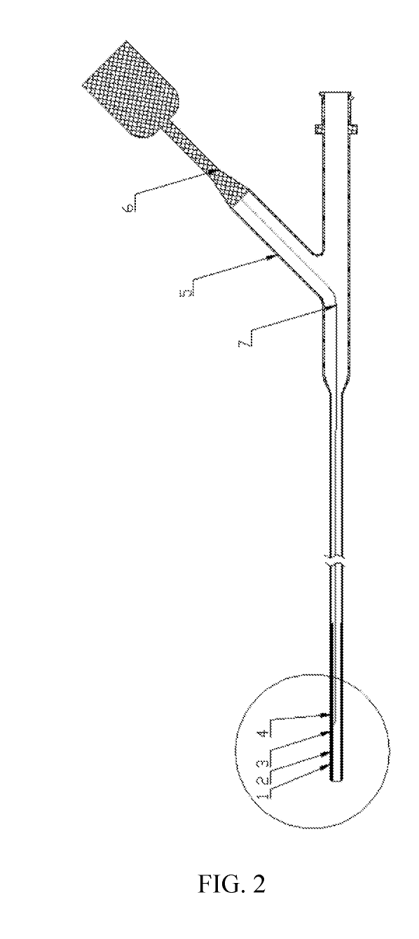

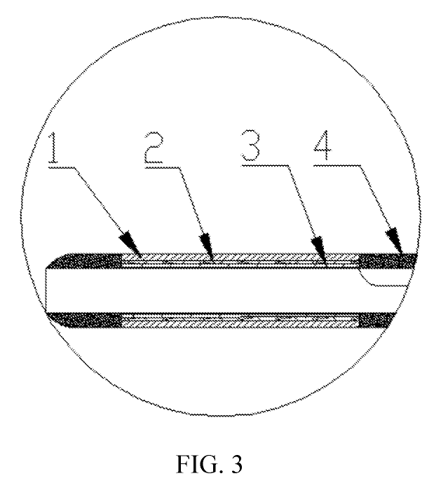

In FIG. 2 and FIG. 3: 1 represents an outer connector, 2 represents a PTC thermistor, 3 represents an inner cable, 4 represents an insulated conduit, 5 represents a Y-shaped three-way insulation tube, 6 represents a cable plug component, and 7 represents an inner cable.

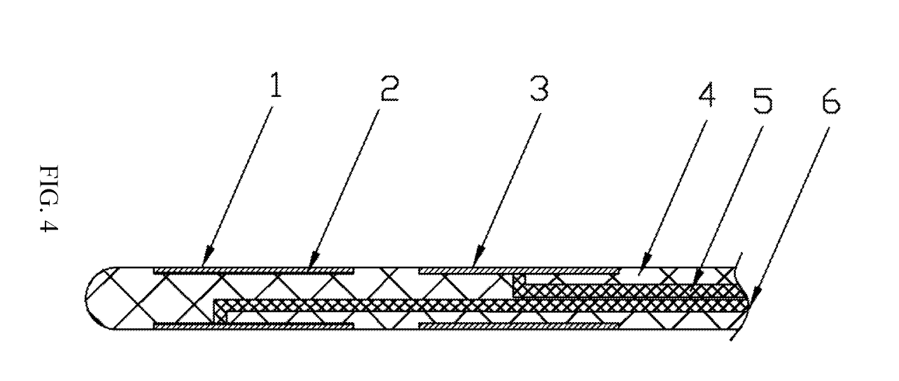

In FIG. 4: 1 represents a first outer connector, 2 represents a PTC thermistor, 3 represents a second outer connector, 4 represents an insulated conduit, 5 represents a first inner cable, and 6 represents a second inner cable.

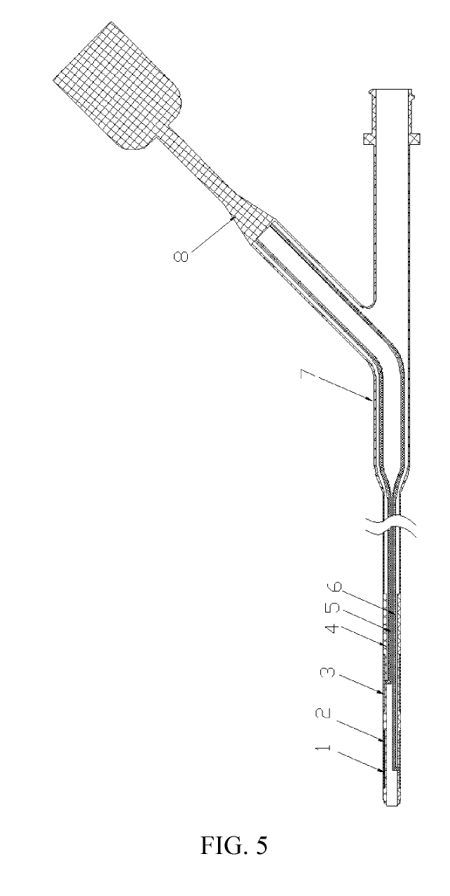

In FIG. 5: 1 represents a first outer connector, 2 represents a PTC thermistor, 3 represents a second outer connector, 4 represents an insulated conduit, 5 represents a first inner cable, 6 represents a second inner cable, 7 represents a Y-shaped three-way insulation tube, and 8 represents a cable plug component.

### DETAILED DESCRIPTION

The present invention is further described below with reference to the accompanying drawings.

Embodiment 1 is shown in FIG. 1.

A radiofrequency ablation catheter includes an electrode ablation head 10, a PTC thermistor 20, and an inner conductor 30, where the electrode ablation head 10 is disposed at an end portion of the inner conductor 30, the PTC thermistor is disposed between the electrode ablation head 10 and the inner conductor 30, and the inner conductor 30 is connected to a cable plug component 40.

The electrode ablation head 10 is a tubular body, and a tail end thereof is smooth and arc-shaped.

A material of the electrode ablation head 10 is 304 medical stainless steel.

A critical temperature of the PTC thermistor 20 is 80° C. to 120° C.

Embodiment 2 is shown in FIG. 2 and FIG. 3.

Another radiofrequency ablation catheter according to the present invention includes an outer connector 1, a PTC thermistor 2, an inner conductor 3, an insulated conduit 4, where the insulated conduit 4 is rod-like, and a tail end thereof is arc-shaped; an outer surface of the insulated conduit 4 is provided with the cylindrical outer connector 1; an inner wall of the outer connector 1 is provided with the PTC thermistor 2; an inner cable 7 is disposed inside the insulated conduit 4; the inner cable 7 and the outer connector 1 are connected inside the insulated conduit 4 through the PTC thermistor 2; the radiofrequency ablation catheter further includes: a cable plug component 6 which is connected to the inner cable 7, where the inner cable 7 is connected to the cable plug component 6 via the interior of a Y-shaped three-way insulation tube 5.

A material of the outer connector 1 is 304 medical stainless steel.

A material of the insulated conduit 4 is polytetrafluoroethylene (PTFE) or Teflon.

Embodiment 3 is shown in FIG. 4.

A radiofrequency ablation catheter includes a first outer connector 1, a PTC thermistor 2, a second outer connector 3, an insulated conduit 4, a first inner cable 5, and a second inner cable 6. The insulated conduit 4 is rod-like, and a tail end thereof is arc-shaped; an outer surface of the insulated conduit 4 is provided with the cylindrical first outer connector 1 and the cylindrical second outer connector 3; an inner wall of the first outer connector 1 is provided with the PTC thermistor 2; the first inner cable 5 and the second inner cable 6 are disposed inside the insulated conduit 4; the second outer connector 3 and the first inner cable 5 are electrically connected inside the insulated conduit 4; and the second inner cable 6 and the first outer connector 1 are connected inside the insulated conduit 4 through the PTC thermistor 2.

Embodiment 4 is shown in FIG. 5.

A therapy device including a radiofrequency ablation catheter includes a cable plug component 8 which is connected to a first inner cable 5 and a second inner cable 6, where the first inner cable 5 and the second inner cable 6 are connected to the cable plug component 8 via the interior of a Y-shaped three-way insulation tube 7.

The PTC thermistors (2) and (20) use a PTC material. The PTC casing is made of nylon 12 (L1940), manufactured by Degussa AG (Germany); superconducting carbon black with an oil absorption value of 780 cm³/100 g, manufactured by Shangdong Zibo Carbon Black Factory; and fumed silica (R106), manufactured by China BlueStar Shenyang Chemical Co. Ltd. The dried and processed nylon 12, carbon black and fumed silica are mixed and processed in a torque rheometer at a temperature of around 190° C. for about 10 minutes. The mixed materials are then conveyed to a mould at a mould temperature of around 200° C. to form PTC sleeves. The pressure is maintained for 2 minutes and the PTC sleeves are allowed to cool down naturally under room temperature. The resultant PTC sleeves have an initial electrical resistance of 1.45×10⁻³ Ω (the electrical resistivity is 1.1×10⁻² Ωcm) at room temperature, and the electrical resistance increases drastically at around 100° C., with a change of a magnitude of around 1×10⁹. Therefore, a low electrical resistance at room temperature and a high resistance change rate can be obtained.

The treatment mechanism of the radiofrequency ablation in the present invention is to heat local tissues in a limited range in contact with the emitter electrode, to denature the local tissues, where heat is rapidly dispersed during conduction to surrounding tissues, and the heat is prevented from spreading to deep tissues. In local radiofrequency ablation, sufficient heat is applied to the vein wall, causing contraction of collagen and exposure of endothelial cells, and as a result, the vein wall thickens, and the luminal contracts and is rapidly organized, forming a fibrous cord; finally, the vein is closed. Since the treatment temperature is limited to a certain temperature, tissue burning, coagulation, vaporization and carbonization are avoided.

In the radio frequency therapy of the present invention, a small incision is made in the back side of the knee, and the catheter is inserted into the vein. A radiofrequency probe attached in the catheter emits energy in the vein, and thus generates heat, causing the vein to shrink and be closed. As the varicosity is caused by backflow of blood in a saphenous vein and its branches, by closing the vein, leg fatigue, pain, itching and other symptoms can be relieved. After receiving the therapy, patients can carry out normal activities immediately. The therapy of endovenous radiofrequency oblation in combination with electrocoagulation for lower extremity varicosity is simple, practical and beautiful, which meets the aesthetic requirement of patients, and is favored by female patients.

The embodiments described above are merely descriptions about preferred implementations of the present invention, but are not intended to limit the concept and scope of the present invention. Various variations and modifications made by persons of ordinary skills in the art to the technical solutions of the present invention without departing from the design concept of the present invention shall fall within the scope of the present invention, and the claimed technical content of the present invention has been fully described in the claims.

What is claimed is:

1. A radiofrequency ablation catheter, comprising an electrode ablation head (10), a Positive Temperature Coefficient (PTC) thermistor (20), and an inner conductor (30), wherein the electrode ablation head (10) is disposed at an end portion of the inner conductor (30), the PTC thermistor is disposed between the electrode ablation head (10) and the inner conductor (30), and the inner conductor (30) is connected to a cable plug component (40).

2. The radiofrequency ablation catheter according to claim 1, wherein the electrode ablation head (10) is a tubular body, and a tail end thereof is smooth and arc-shaped.

3. The radiofrequency ablation catheter according to claim 1, wherein a material of the electrode ablation head (10) is 304 medical stainless steel.

4. The radiofrequency ablation catheter according to claim 1, wherein a critical temperature of the PTC thermistor (20) is 80° C. to 120° C.

5. A radiofrequency ablation catheter, comprising an outer connector (1), a Positive Temperature Coefficient (PTC) thermistor (2), an inner conductor (3), an insulated conduit (4), wherein the insulated conduit (4) is rod-like, and a tail end thereof is arc-shaped; an outer surface of the insulated conduit (4) is provided with the cylindrical outer connector (1), an inner wall of the outer connector (1) is provided with the PTC thermistor (2), and an inner cable (7) is disposed inside the insulated conduit (4); the inner cable (7) and the outer connector (1) are connected inside the insulated conduit (4) through the PTC thermistor (2); further comprising: a cable plug component (6) which is connected to the inner cable (7), wherein the inner cable (7) is connected to the cable plug component (6) via the interior of a Y-shaped three-way insulation tube (5).

6. The radiofrequency ablation catheter according to claim 5, wherein a material of the outer connector (1) and the inner conductor (3) is 304 medical stainless steel.

7. The radiofrequency ablation catheter according to claim 6, wherein a material of the insulated conduit (4) is polytetrafluoroethylene (PTFE) or Teflon.

8. A therapy device comprising the radiofrequency ablation catheter of claim 5, comprising a first outer connector (1), a Positive Temperature Coefficient (PTC) thermistor (2), a second outer connector (3), an insulated conduit (4), a first inner cable (5), and a second inner cable (6), wherein the insulated conduit (4) is rod-like, and a tail end thereof is arc-shaped; an outer surface of the insulated conduit (4) is provided with the cylindrical first outer connector (1) and the cylindrical second outer connector (3) respectively; an inner wall of the first outer connector (1) is provided with the PTC thermistor (2); the first inner cable (5) and the second inner cable (6) are disposed inside the insulated conduit (4); the second outer connector (3) and the first inner cable (5) are electrically connected inside the insulated conduit (4), and the second inner cable (6) and the first outer connector (1) are connected inside the insulated conduit (4) through the PTC thermistor (2).
